(19) United States
(12) Patent Application Publication
(10) Pub. No.: US 2021/0094891 A1
(43) Pub. Date: Apr. 1, 2021

(54) 1-STEP RADIOSYNTHESIS OF [18F]SFB

(71) Applicants: RIGSHOSPITALET, Copenhagen O (DK); University of Copenhagen, Copenhagen K (DK)

(72) Inventors: Andreas KJÆR, Frederiksberg (DK); Matthias Manfred HERTH, Malmo (SE); Jacob MADSEN, Skovlunde (DK); Ida Nymann PETERSEN, Kongens Lyngby (DK)

(21) Appl. No.: 17/045,532

(22) PCT Filed: May 27, 2019
(86) PCT No.: PCT/EP2019/057771

§ 371 Date: Oct. 6, 2020

(30) Foreign Application Priority Data
Apr. 6, 2018 (DK) PA 2018 70204

(51) Int. Cl. C07B 59/00; C07D 407/12; C07D 207/46

(52) U.S. Cl. CPC C07B 59/002; C07B 2200/05; C07D 207/46; C07D 407/12

(57) ABSTRACT

There is provided a synthesis of [¹⁸F]SFB (N-succinimidyl 4-[¹⁸F]fluorobenzoate) using a one-step reaction procedure without generating radioactive waste gases. [¹⁸F]SFB is useful as a reagent for labeling of low- and high-molecular weight compounds such as peptides and antibodies which can then be used for PET (Positron Emission Tomography) diagnostic studies.

### FIELD OF THE INVENTION

The present invention relates to the synthesis of [¹⁸F]SFB (N-succinimidyl 4-[¹⁸F]fluorobenzoate) using a one-step reaction procedure without generating radioactive waste gases. [¹⁸F]SFB is useful as a reagent for labeling of low- and high-molecular weight compounds such as peptides and antibodies which can then be used for PET (Positron Emission Tomography) diagnostic studies.

### BACKGROUND OF THE INVENTION

Fluorine-18 is a very attractive radionuclide for PET imaging because it can be produced in amounts that allow commercialization. Fluorine-18 has also outstanding nuclear diagnostic imaging properties such as high-spatial resolution. Furthermore, it results in a low and acceptable radiation burden for molecular imaging purposes. Its high positron abundance and nearly monochromatic emission lead to simplified detection, data processing and greater sensitivity. Fluorine-18 is also preferred for the development of novel PET tracers because it is available in high specific activity. The flexibility of fluorine-18 chemistry not only produces large amounts of useful PET tracers originated from small organic molecules but also has potential to turn certain highly-specific targeting biological molecules, such as proteins or peptides into valuable PET tracers.

The concept of applying radiolabeled biomolecules to target receptor-(over)expressed tissues in vivo has opened up a new avenue for PET as a very useful diagnostic tool to visualize for example tumor lesions. However, because of the harsh chemical conditions associated with direct radio-fluorination that is usually not compatible with most biological samples, the incorporation of radionuclide-tagged prosthetic groups into biomolecules becomes the method of choice.

Indirect labeling methods using prosthetic groups that use mild labeling conditions are as such an interesting alternative. Succinimidyl-4-[¹⁸F]-fluorobenzoate ([¹⁸F]SFB) is an optimal reagent (prosthetic group) for such purpose and can be used to label proteins, peptides, nanomedicines and small molecules with fluorine-18 because of good conjugation yields and metabolic stability. It is widely used within the nuclear medicine community.

The most frequently applied clinically relevant synthesis of [¹⁸F]SFB makes use of a 3-step synthesis procedure and requires multiple SPE- or HPLC-purifications. 3-step radioactive synthesis procedures are usually disadvantaged because they need special and dedicated automatization. Not all synthesis modules support 3-step synthesis procedures and as such the synthesis of [¹⁸F]SFB cannot easily be implemented at all radiopharmaceutical productions sites. The 3-step procedure also results in volatile radioactive by-products that are released during the synthesis. Volatile radioactive substances which are removed by the ventilation system should be minimized. As such, further improvement and simplifying [¹⁸F]SFB synthesis would be very desirable. It is therefore an object of the present invention to simplify operations for [¹⁸F]SFB synthesis, reduce the synthesis time, to improve reaction efficiency and reduce volatile by-products.

### SUMMARY OF THE INVENTION

A new, one-step reaction route to synthesize [¹⁸F]SFB, which avoid formation of volatile radioactive by-products was developed. The new synthesis of [¹⁸F]SFB needs less synthesis time and results in comparable radiochemical yields and radiochemical purity compared with routinely applied procedures. As such, this invention simplifies the production of [¹⁸F]SFB.

In a first aspect of the present invention there is provided process for preparing [¹⁸F]SFB comprising:

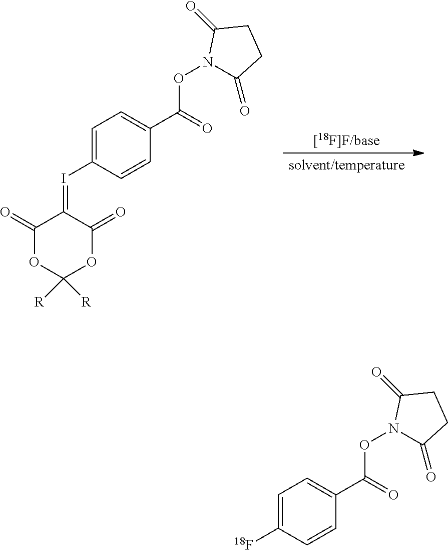

wherein R is methyl, ethyl, propyl or closed into six and seven carbons rings or adamantan.

In a preferred embodiment the reaction is as follows:

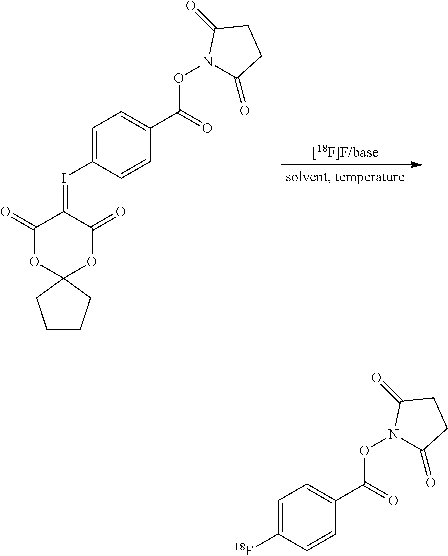 [SMILES: O=C1OC2(CCCC2)OC(=O)C1=Ic1ccc(C(=O)ON2C(=O)CCC2=O)cc1 ; O=C(ON1C(=O)CCC1=O)c1ccc([18F])cc1]

wherein the process is performed in the presence of carbonate ions by reacting 2,5-dioxopyrrolidin-1-yl-4-((7,9-dioxo-6,10-dioxaspiro[4.5]decan-8-ylidene)-13-iodaneyl)benzoate with a dried [¹⁸F]fluoride to give [¹⁸F]SFB.

In a second aspect of the present invention there is provided spirocyclic iodonium precursors of SFB (compound of formula (I)):

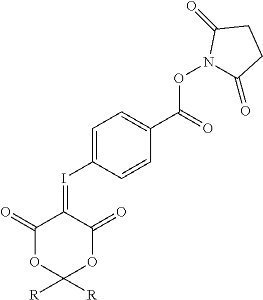

where R is methyl, ethyl, propyl or closed into six and seven carbons rings and/or adamantan.

### BRIEF DESCRIPTION OF THE DRAWINGS

FIG. 1 shows the reaction scheme used to prepare [¹⁸F]SFB in accordance with the present invention.

FIG. 2 shows a liquid HPLC chromatogram illustrating an analysis of [¹⁸F]SFB prepared in accordance with the present invention.

FIG. 3 shows the gamma-radioactivity and UV HPLC profiles of the reaction mixture of [¹⁸F]SFB prepared in accordance with the present invention.

FIG. 4 shows the gamma-radioactivity and UV HPLC profiles of the reaction mixture of [¹⁸F]SFB prepared in accordance with the present invention spiked with non-radioactive SFB reference compound standard.

FIG. 5 shows the ¹H,¹³C-NMR of one precursor of the present invention.

FIG. 6 shows ¹H-NMR of an alternative precursor of the present invention.

### DETAILED DESCRIPTION OF THE INVENTION

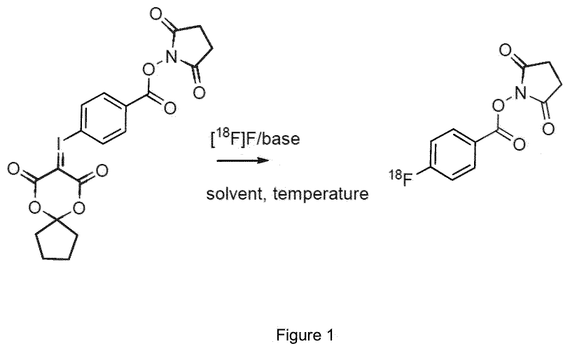

In this invention, a novel one-step procedure is presented using new precursors that are based on spirocyclic iodonium ylides. The radiosynthesis procedure incorporating features of the present invention follows the procedure depicted in the reaction scheme in FIG. 1 showing the reaction of the precursor with [¹⁸F]FK to give [¹⁸F]SFB. In the presence of base, the precursor is reacted under heating for 4 minutes with the dried [¹⁸F]fluoride. [¹⁸F]SFB can be purified by different types of SPE or semipreparative HPLC. After evaporation of the solvent, [¹⁸F]SFB is dissolved in an aqueous buffer and a solution of the peptide/antibody/protein/small molecule is added for labeling. The radiotracer is purified via reversed phase HPLC on a standard semi-preparative C18 column (or a SEC column) and afterwards separated with standard solid-phase extraction.

A novel feature of the described radiosynthesis of [¹⁸F]SFB is that it is a one-step synthesis and enormously reduces its overall complexity. The simpler synthesis is much easier to automate and can thus be implemented on almost all existing automatization devices. The precursor of the present invention is stable at 0° C., and the overall synthesis time is faster. In addition, the use of this procedure does not result in the formation of radioactive volatile side products as it is the case for the usually applied 3-step synthesis. Moreover, the overall synthesis time is shortened. The synthesis procedure of the present invention leads to moderate RCYs and to a radiochemical purity which are at least comparable to those described in the literature for prior procedures.

The improved and concise synthesis makes routine production of [¹⁸F]SFB much more practical and attractive. It is believed that biomedical discovery and clinical studies using ¹⁸F-labeled biomolecules as a research tool will accelerate if [¹⁸F]SFB can more widely be used as its availability increases. A great number of biologists and clinicians could benefit from the ability to incorporate ¹⁸F-labeling onto a variety of biomolecules and having access to this synthesis route of [¹⁸F]SFB.

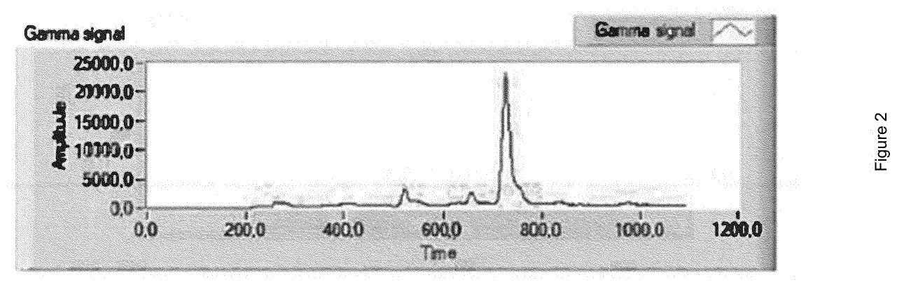

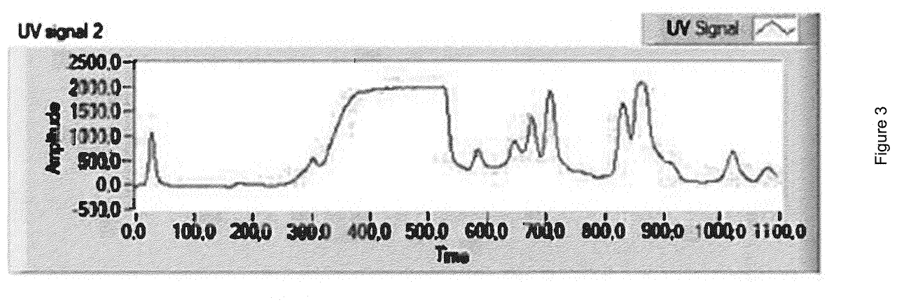

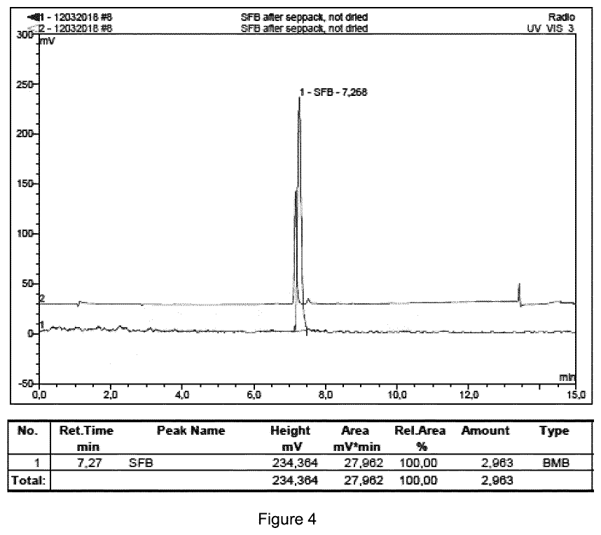

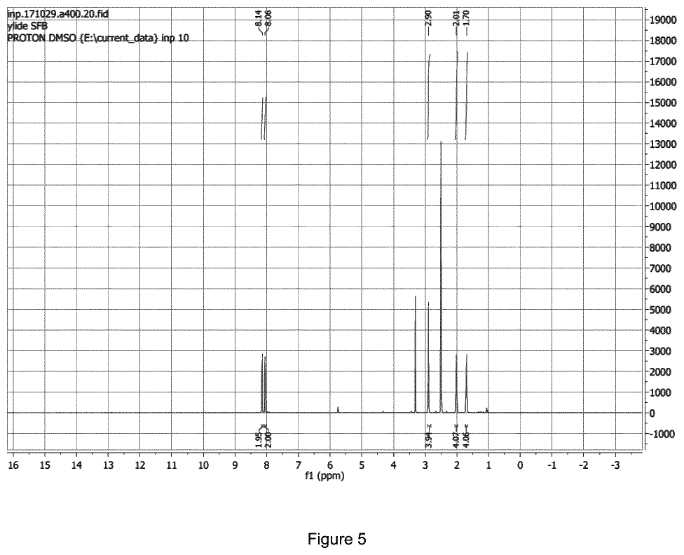

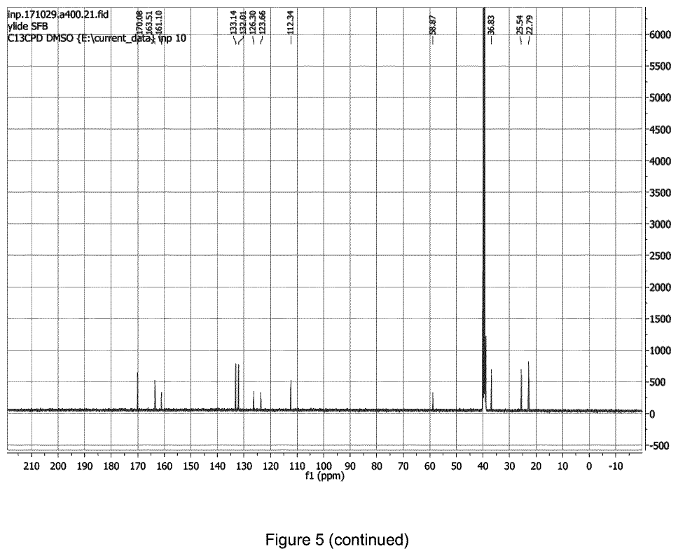

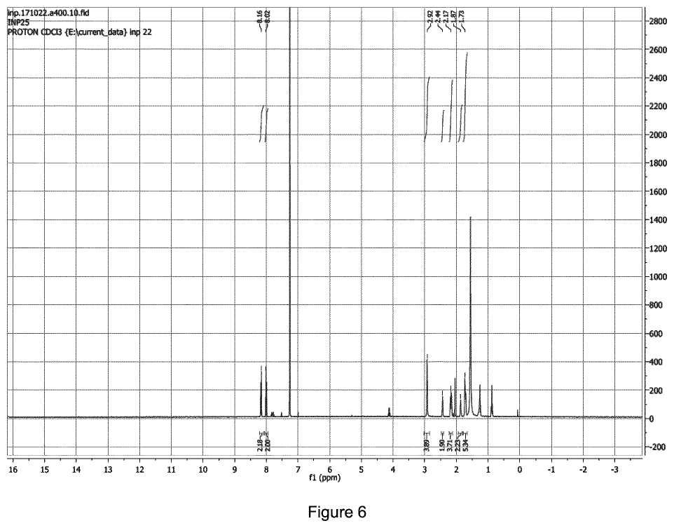

The HPLC diagram of FIGS. 2, 3 and 4 shows typical chromatograms using the synthesis in accordance with the present invention. Analytical HPLC chromatograms have been obtained with C18 LUNA (phenomenex) column, 250×4.6 mm in 2 mL/min solvent flow. A gradient system with two eluents, A and B, was used, with the fraction of B v. FIG. 5 shows the NMR of corresponding spirocyclic iodonium ylide precursors. FIG. 6 shows HNMR of one alternative precursor, 2,5-dioxopyrrolidin-1-yl 4-((4′,6′-dioxospiro[tricyclo[4.4.0.03,8]decane-4,2′-[1,3]dioxan]-5′-ylidene)-13-iodaneyl)benzoate. More specifically FIG. 2 shows a semi preparative chromatogram, FIG. 3 shows a UV-chromatogram, while FIG. 4 shows a spiked chromatogram of purified [18F]SFB. In FIG. 5 there is shown a H-NMR spectrum of the precursor, 2,5-dioxopyrrolidin-1-yl 4-((7,9-dioxo-6,10-dioxaspiro[4.5]decan-8-ylidene)-13-iodaneyl)benzoate.

1. A process for preparing [¹⁸F]SFB comprising: 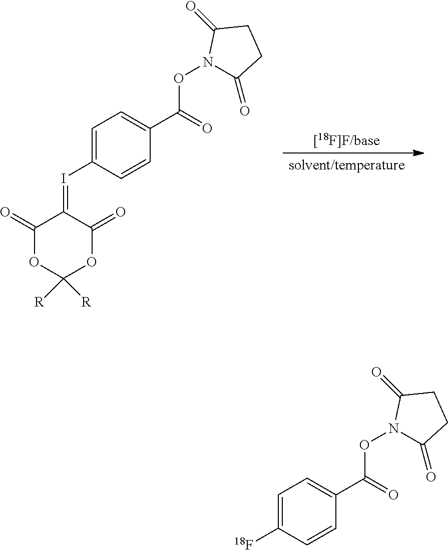
wherein R is methyl, ethyl, propyl or closed into a six or seven carbons ring or adamantan.

2. The process according to claim 1, wherein the process is performed in the presence of a base selected from carbonate base, hydrogen carbonate, triethylamine, DIPEA, and DBU.

3. The process according to claim 1, wherein the solvent is selected from: DMF, DMSO or MeCN or similar polar, aprotic solvents

4. The process according to claim 1, wherein the process is performed in the presence of a base selected from carbonate base, hydrogen carbonate, triethylamine, DIPEA, and DBU by reacting 2,5-dioxopyrrolidin-1-yl-4-((7,9-dioxo-6,10-dioxaspiro[4.5]decan-8-ylidene)-13-iodaneyl)benzoate with a dried [¹⁸F]fluoride to give [18F]SFB.

5. A spirocyclic iodonium precursor of SFB comprising: 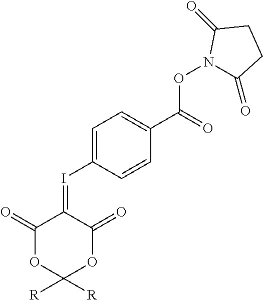
wherein R is methyl, ethyl, propyl or closed into a six and seven carbons ring or adamantan.

6. The spirocyclic iodonium precursor of claim 5, wherein the precursor is 2,5-dioxopyrrolidin-1-yl-4-((7,9-dioxo-6,10-dioxaspiro[4.5]decan-8-ylidene)-13-iodaneyl)benzoate
